(19) United States
(12) Patent Application Publication
(10) Pub. No.: US 2019/0231389 A1
(43) Pub. Date: Aug. 1, 2019

(54) ACCESS TROCAR, METHOD FOR USING AN ACCESS TROCAR, KIT COMPRISING AN ACCESS TROCAR AND A GUIDEWIRE

(71) Applicants: Sociedade Beneficente Israelita Brasileira Hospital Albert Einstein, São Paulo (BR); ITM S/A Indústria de Tecnologias Médicas, Canoas (BR)

(72) Inventors: Emerson Rodrigo Primaz, Novo Hamburgo (BR); Dimitrios Georgiadis, Porto Alegre (BR); Denise Araújo Lapa Pedreira, Vila Mariana (BR)

(21) Appl. No.: 16/324,093

(22) PCT Filed: Aug. 7, 2017
(86) PCT No.: PCT/BR2017/050223

§ 371 Date: Apr. 24, 2019

(30) Foreign Application Priority Data
Aug. 9, 2016 (BR) BR2020160183702

(51) Int. Cl. A61B 17/34

(52) U.S. Cl. CPC A61B 17/3423; A61B 17/3498; A61B 2017/00557; A61B 2017/3486; A61B 17/3474

(57) ABSTRACT

The present invention concerns an access trocar (1) comprising: a cannula (20) equipped with a hollow portion (25) and with two ends (20′, 20″), where a first end of the cannula (20) is a free end (20′) and the opposite end is an end for connection (20″) to a handling portion (40) through a retention valve (12) and a sheath (10).

The handling portion (40) comprises a rod (31) that is connectable to the hollow portion of the cannula (20) through the connecting end (20′); the cannula (20) also comprises an inflatable balloon (22) and a circlip (21) equipped with internal orifices for insertion regarding the cannula (20), where the inflatable balloon (22) is also connected to the retention valve (12) through a duct (23) connected to the cannula (20). It also describes a method for using an access trocar (1) and a kit comprising an access trocar (1) and a guidewire (45).

The present invention concerns an access trocar. More specifically, a trocar that uses a guidewire to position the trocar. The present invention also concerns a method for using an access trocar, a kit comprising an access trocar and the use of a guidewire.

### DESCRIPTION OF THE STATE OF THE ART

Laparoscopy and myelomeningocele procedures require operating on a fetus when it is still in the intrauterine state, which is to say, inside the mother's uterus. For this reason, these procedures are performed with the help of appropriate instruments and apparatus so that it is possible to visualize the abdominal cavity without the need for major incisions and, consequently, with less surgical trauma.

For this purpose, the trocar was invented, which is a valved instrument that allows for the performance of surgical procedures such as those mentioned previously.

Trocars can present variable diameters, in accordance with the diameters of the forceps to be used, being basically composed of two parts: an external portion which possesses a channel for the introduction of the forceps with a valved system which prevents the escape of fluid and an internal portion equipped with a cutting element to facilitate the introduction of the trocar.

Additionally, for the performance of intrauterine procedures such as myelomeningocele surgery, it is necessary to use an access trocar equipped with special characteristics which facilitate its insertion and removal from the body of the patient, without the fetus being affected.

In this regard, the state of the art presents various proposals which encompass constructive arrangements applied to trocars, where the following documents may be highlighted:

Document U.S. 6,524,283 describes a balloon anchor which allows for the fixing of a surgical instrument, such as a conventional trocar sheath, inside an aperture formed by a trocar puncture. When used in a trocar sheath, the anchor is fixed to the smooth exterior surface of the sheath for extension through the aperture of the puncture, where adhesive or mechanical means are provided for fixing the balloon to the instrument. No modification to the structure of the instrument is necessary.

While document CA 2.845.401 describes a support device for a trocar which extends through a wall of the patient's body, including an inflatable collar which extends around the cited trocar, where said collar may be inflated to a predetermined size using a fluid source located on the trocar support.

This source comprises a pump mechanism for supplying a fixed volume of fluid which allows inflation only to a given size of the balloon. A back rest is also mentioned configured to be positioned on an external surface of the trocar sleeve and longitudinally adjustable to be located in a given position.

Document CA 2.904.686 describes a cannula with a retention element and a method of manufacturing the cannula set. In some forms of realization, the body of the cannula may include one or more fluid channels or grooves which extend longitudinally, generally from the fluid inlet port to the distal end of the cannula.

The fluid channel may be formed on the external surface of the body of the cannula and extend to a given depth inside the body. Thus, the fluid channel allows the passage of fluid to the inflation and deflation balloon. In an advantageous manner, with the fluid channel embedded in the body of the cannula, the sleeve set may have a relatively small external diameter.

The trocars existent in the state of the art have the disadvantage that positioning and fixing them in the body of the patient is laborious and, in many cases, ends up causing lesions. So there is a gap in the state of the art regarding access trocars and the consequent methodology which allows for the easy and correct positioning of the trocar in the patient's body.

### OBJECTIVES OF THE INVENTION

A first objective of the present invention is to provide an access trocar capable of receiving a guidewire and which directs the trocar to its access point in the tissue of the patient's skin.

A second objective of the present invention is to eliminate the use of external tubes in the inflation and deflation system.

A third objective of the present invention is to improve the locking and facilitate the positioning of the trocar in the patient's body.

An additional objective of the present invention consists of an access trocar capable of being positioned in the skin of a patient through the use of a guidewire.

The present invention also has the objective of providing a method of using an access trocar and a kit comprising an access trocar and a guidewire.

An additional objective of the present invention is to provide the use of a guidewire for positioning an access trocar.

### BRIEF DESCRIPTION OF THE INVENTION

The objectives of the present invention are achieved through an access trocar, the access trocar comprising: a cannula equipped with a hollow portion and two ends, where a first end of the cannula is a free end and the opposite end is an end for connection to a handling portion through a sheath.

The handling portion comprises a rod that is connectable to the hollow portion of the cannula through the connecting end, the cannula also comprises an inflatable balloon and a circlip equipped with internal orifices for insertion into the cannula, where the inflatable balloon is also connected to the retention valve through a duct connected to the cannula.

The present invention also describes a method for using an access trocar and a kit comprising an access trocar.

### BRIEF DESCRIPTION OF THE FIGURES

The present invention shall be described in more detail below based on an example of execution represented in the drawings. The figures show:

FIG. 1 is a perspective view of the access trocar proposed in the present invention;

FIG. 2 is an upper view of the access trocar proposed in the present invention;

FIG. 3 is a side view of the cannula of the access trocar proposed in the present invention;

FIG. 4 is a side view of the cannula and the sheath which form part of the access trocar proposed in the present invention;

FIG. 5 is a representation of the access trocar and the guidewire used to position the trocar in the epithelial tissue of a patient;

FIG. 6 is a cross-section representation of the access trocar proposed in the present invention; and

FIG. 7 is an additional upper view of the access trocar proposed in the present invention.

### DETAILED DESCRIPTION OF THE FIGURES

The present invention concerns an access trocar 1 (trocar 1). More specifically, a trocar 1 which uses a guidewire 45 for its due positioning.

The reference to an access trocar 1 must be understood as a trocar 1 capable of being positioned in a region of the patient's body using a guidewire 45, such as shall be described below.

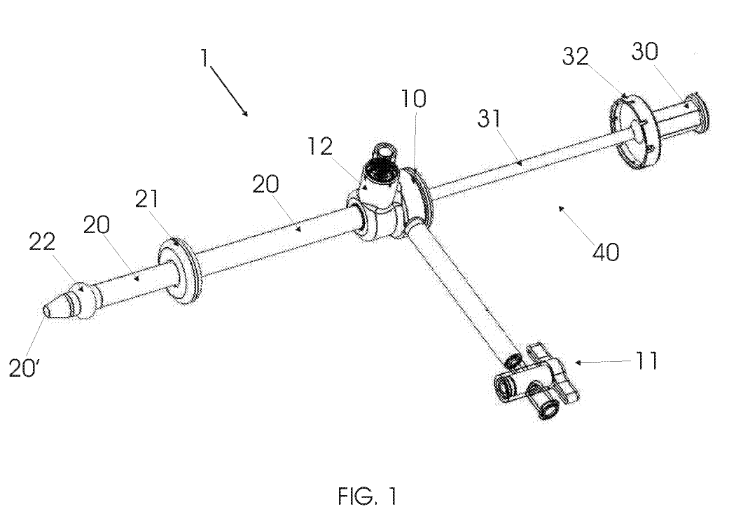

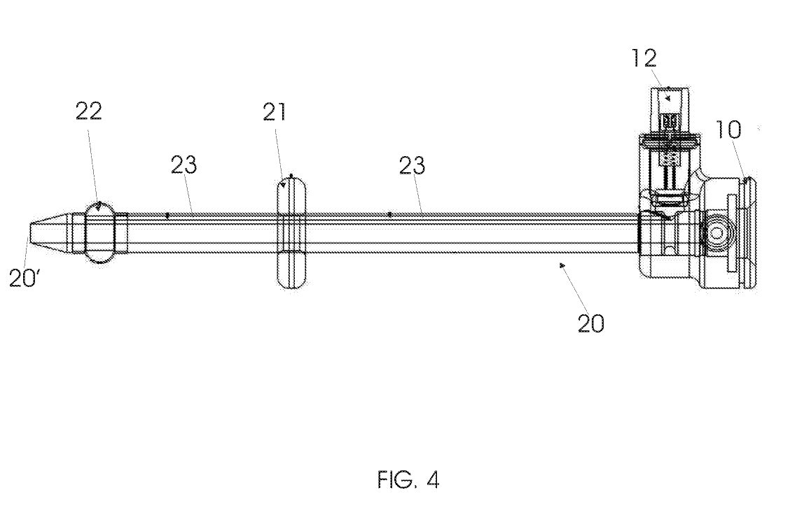

With reference to FIGS. 1 to 7, note that the trocar 1 proposed in the present invention basically comprises a cannula 20 connected to a handling portion 40.

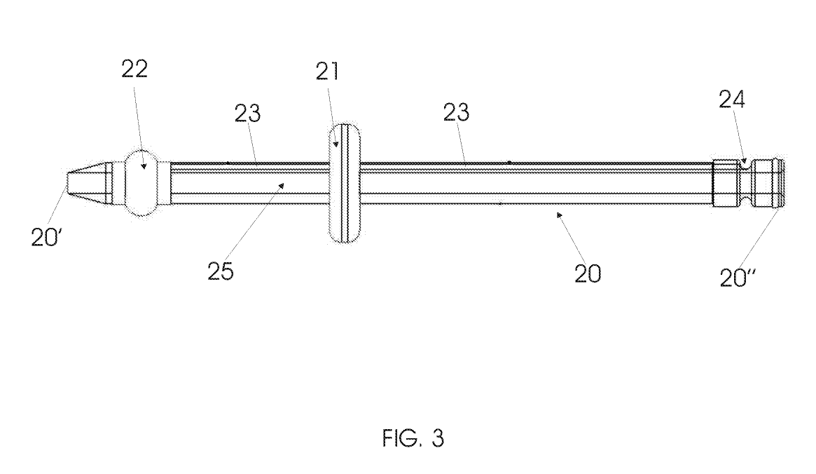

The cannula 20 consists of a rigid structure preferably of a cylindrical shape and equipped with a hollow portion 25. More specifically, the cannula 20 has the function/objective of providing support to the other components of the trocar 1, as shall be described below. FIG. 3 is a representation of the cannula 20 used in the trocar 1 of the present invention.

In this preferred configuration of the trocar 1, note that the cannula 20 comprises two ends 20′ and 20″, consisting of a free end 20′ and a second end 20″ described as a connecting end 20″.

In another preferred mode, the free end 20′ possesses the geometry of a cone cut by a plane parallel to the base, as illustrated in FIG. 1.

In other words, the cited cut cone shape of the free end 20′ comprises a cone lacking a tip, such that it possesses a smaller circumferential base parallel to a larger circumferential base, connected by a curved surface, thus, characterizing a cut cone.

In FIGS. 1 to 7, note also that the connecting end 20″ allows the connection of the cannula 20 to the handling portion 40 of the trocar 1.

More specifically, the handling portion 40 basically comprises a rod 31, a dilator structure 30 and a circular structure 32. The handling portion 40 is configured so that the internal diameter of the trocar 1 is compatible with the diameter of a guidewire 45, such that the guidewire 45 can be inserted in the trocar 1 with the help of the cited handling portion 40.

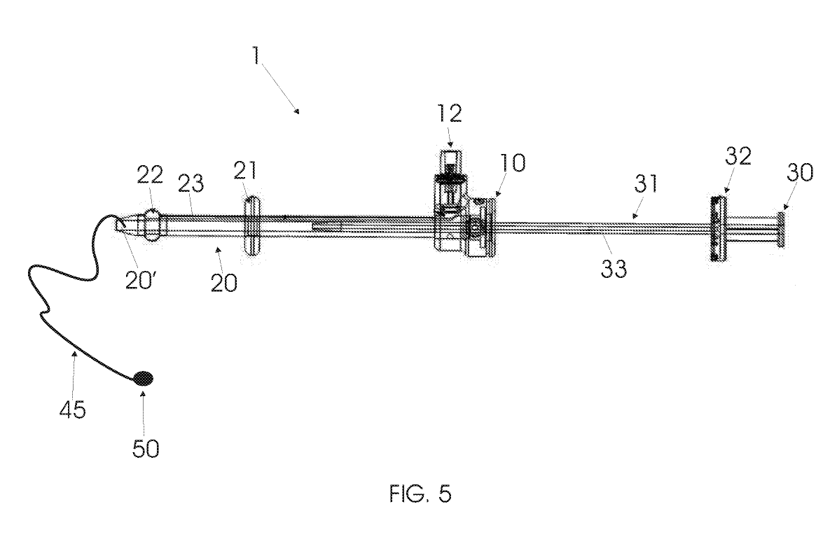

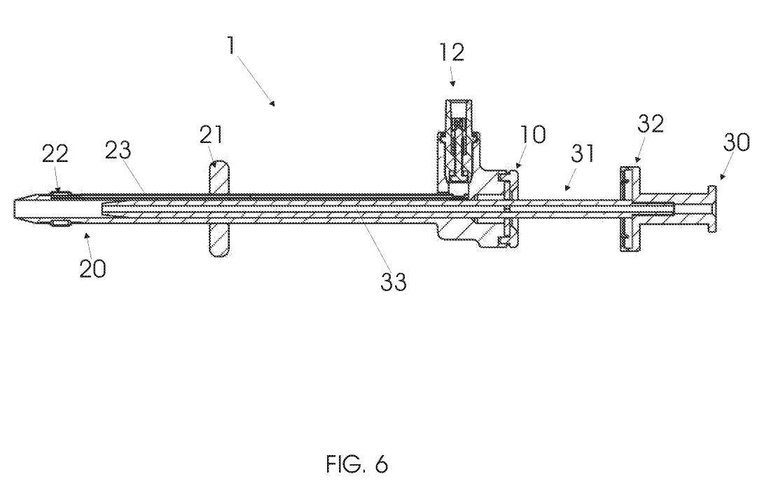

So, and in reference to FIGS. 5 and 6, “compatible” should be understood to mean the characteristic of the trocar 1 of comprising a guide-wire 45 which can be inserted through its interior, using the rod 31 and an orientation duct 33, which is to say, the diameter of the guidewire 45 must be slightly smaller than the diameter of the orientation duct 33, as shall be described in greater detail below. Obviously, it is also understood that the guidewire 45 is also capable of being inserted through the hollow portion 25 of the cannula 20.

With reference to the rod 31, this must be understood as a cylindrical duct with a smaller diameter than the diameter of the cannula 20. So, it is understood that, as a function of the diameters of the cannula 20 and the rod 31, the rod 31 may be introduced into the hollow portion 25 of the cannula 20, as represented in FIGS. 5 and 6.

It is also highlighted that the cylindrical configuration of the rod 31 must be understood only as a preferential characteristic of the present invention, and must not be understood as a limiting characteristic thereof.

Regarding the dilator structure 30, this must be understood as a handle portion of the trocar 1, which is to say a portion of the trocar 1 capable of being handled by the medical professional who operates the object of the present invention.

So, it is understood that the handling of the dilator structure 30 by the medical professional allows for the introduction and consequent transference of the rod 31 into the cannula 20.

Regarding the circular structure 32, this element has the function of locking the handling portion 40 to the body of the trocar 1 on the connection of the structure 32 to the sheath 10; so, it is understood that the circular structure 32 envelops the sheath 10 when the rod 31 is introduced into the hollow portion 25 of the cannula 20.

With reference to FIGS. 1 to 7, note that the connection of the cannula 20 to the handling portion 40 occurs through the cited sheath 10. In this regard, the sheath 10 must be understood as a rigid structure equipped with holes which allow for its connection to the rod 31 of the handling portion 40, with a retention valve 12 and also the cannula 20. In one configuration, the arrangement of the cannula 20 and the sheath 10 in a single piece is wholly acceptable.

With reference to the retention valve 12, this is configured to allow the insufflation/desufflation of the inflatable balloon 22 through the use of a fluid.

Said retention valve 12 is configured to direct the fluid flow, which is to say, the function of the valve 12 is to ensure that the direction of the transfer of the fluid used in the positioning of the trocar 1 is only in the direction from the external source to the trocar 1 at the time of the insufflation of the balloon 22, and the from the inflatable balloon to the external environment, at the time of its desufflation (emptying of the balloon 22).

The trocar 1 proposed also comprises a fluid insufflation valve 11, hereafter also referred to only as a valve 11, which comprises a structure selectively activated by an operator, such as a medical professional who handles the trocar 1 that is the object of the present invention. Depending on the application of the trocar 1, the valve 11 may be used to introduce a given fluid into the cannula 20, for example, the valve 11 may be used to insert and direct a given gas into the uterus of a patient through the cannula 20.

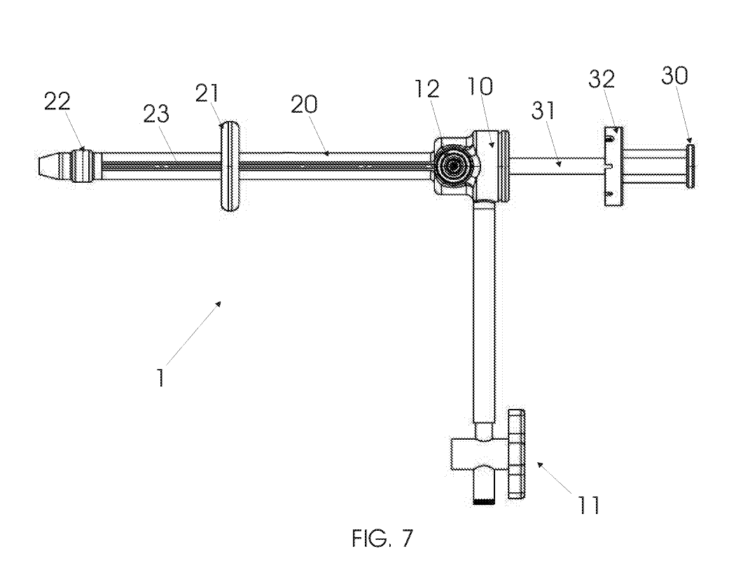

With preferential reference to FIGS. 1 and 7, note that the cannula 20 which forms part of the trocar 1 of the present invention also comprises an inflatable balloon 22 and a circlip 21.

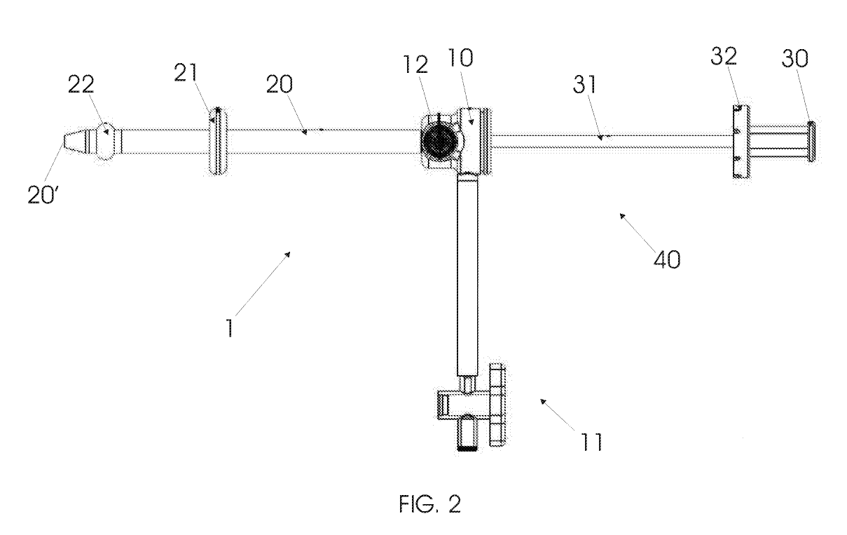

The inflatable balloon 22 and the circlip 21 surround the whole circumference of the cannula 20, however, and as observed in FIGS. 1 and 2, both the inflatable balloon 22 (balloon 22) and the circlip 21 envelop only part of the length of the cannula 20.

So, and as can be seen principally in FIGS. 1 and 2, most of the length of the cannula 20 is not enveloped by the balloon 22 and circlip 21.

Additionally, it is observed that the balloon 22 is positioned close to the free end 20′ of the cannula 20, while the circlip 21 is positioned on an intermediate portion between the cited balloon 22 and the retention valve 12.

More specifically, the circlip 21 comprises a structure of a substantially circumferential and flat geometry, possessing an internal orifice at its center, said orifice having a diameter compatible with the external diameter of the cannula 20, so that it can be enveloped by the circlip 21 and consequently the circlip 21 can be duly positioned and fixed to the cannula 20, as illustrated in the figures.

In an alternative configuration, the circlip 21 may possess other shapes, such as triangular, rectangular, hexagonal, trapezoidal, and spherical, among others, provided that they provide adequate support and fixing to the trocar 1 when it is used in surgical procedures.

So, it is highlighted that the representation of the shape of the circlip 21 as shown in the figures must be understood as a merely preferential representation of the present invention.

Regarding the inflatable balloon 22, this preferably comprises an internal orifice in its central portion thus allowing for the insertion of the cannula 20.

Additionally, the inflatable balloon 22 must preferably be manufactured from an expandable material, thus allowing cited balloon 22 to inflate/deflate through the respective introduction/removal of the fluid from the retention valve 12.

In this regard, and as better observed in FIGS. 3 and 6, it is highlighted that the connection between the inflatable balloon 22 and the retention valve 12 occurs through a duct 23 connected to the cannula 20. In one configuration, said duct 23 may be positioned in the hollow portion 25 or on the upper surface of the cannula 20. More specifically, any positioning of the duct 23 is acceptable, provided that it allows for the connection between the retention valve 12 and the inflatable balloon 22.

The duct 23 consists of a channel of preferably cylindrical geometry, like the cannula 20. However, one preferential configuration requires that the duct 23 has a smaller external diameter than the internal diameter of the cannula 20. In an alternative form, the duct 23 is made with other geometries, such as rectangular or triangular, for example.

With a view to ensuring the correct connection between the duct 23 and the inflatable balloon 22, note that in FIG. 3 the cannula 20 comprises at its connecting end 20″ a groove 24 which allows the fluid to be conducted from its external source of fluid emission, passing through the retention valve 12 and arriving at the inflatable balloon 22, thus crossing the internal area of the duct 23.

Following the characteristics which form the trocar 1 proposed in the present invention, the mechanical coupling of the sheath 10 to the insufflation valve 11 occurs by means of a docking system. Similarly, the sheath 10 is also coupled by a fitting to the valve 12.

One preferential configuration includes using an interference fit, such as a press fit, to couple the insufflation valves 11 and retention valves 12 to the cited sheath 10.

To better fix the coupling of the valves, a layer of adherent substance is applied, such as glue. Ideally UV Loctite 3311 glue is used, which is specific to medical instruments. However, it is emphasized that the use of the cited UV glue must only be understood as a preferential characteristic of the present invention, and must not be considered as a limiting characteristic thereof.

So, the cited fittings are achieved in such a way as to allow a hermetic seal which allows for the sealing of the fittings so that there is no leakage of fluid or other substances from the trocar to the external environment or entry of fluids or other substances from the external environment to the inside of the trocar.

It is also possible that this fitting is achieved by other means, such as the insertion of threads in the parts to be coupled, as well as welding or gluing. However, it is essential that the fittings provide an adequate seal to prevent the contamination of the fluid in the interior, or the leakage of fluid to the external environment.

As previously observed, the supply of fluid occurs continuously when the retention value 12 is opened, which allows the fluid to flow from the external source to the balloon 22, travelling through the duct 23 positioned in the cannula 20.

This arrangement adopted for the trocar 1 proposed in the present invention ensures that advantages related to the use of this element are achieved.

More specifically, and due to the fact that it possesses the free end 20′ of the cannula 20 and the duct 23, rod 31 and orientation duct 33, the trocar 1 proposed in the present invention allows for the use of the guidewire 45 to enable its correct fixing and positioning in the body of the patient, as shall be described in greater detail below. A merely preferential representation of the guidewire 45 is illustrated in FIG. 5.

In this configuration of the invention, the guidewire 45 used preferably possesses a diameter in the range of 0.70 mm to 1.0 mm (millimeters) and ideally in the range of 0.85 mm to 1.0 mm. Regarding its length, this is preferably located at 450 mm, but clearly this value may vary depending on the use intended for the trocar 1.

These dimensions allow the guidewire 45 to be duly used to ensure the correct positioning of the trocar 1, thus allowing the guidewire 45 to be inserted through the inside of the cannula 20, of the rod 31 and of its orientation duct 33.

In this regard, it is highlighted that the diameter of the orientation duct 33 is preferably in the range of 1.0 mm to 2.0 mm and ideally in the range of 1.0 mm to 1.5 mm. As previously observed, it is understood that the diameter of the orientation duct 33 should be slightly greater than the diameter of the guidewire 45, thus allowing the guidewire to be able pass through the inside of the duct 33. In a general sense, it is highlighted that the diameter of the orientation duct 33 is, at most, twice that of diameter of the guide-wire 45.

Concerning the diameter of the cannula 20, this must obviously be greater than the diameter of the guidewire 45 and the orientation duct 33. As such, during the insertion of the guidewire 45 through the trocar 1, a kind of “funnel” will be configured between the cannula 20 and the orientation duct 33, thus ensuring that the guidewire 45 is directed to the inside of the duct 33 and that consequently the trocar 1 can be duly positioned in the patient, as will be described below. In general sense, it is proposed that the diameter of the cannula 20 is a maximum of seven times greater than the diameter of the orientation duct 33.

Furthermore, regarding the guidewire 45, this is made from malleable material, preferably metal, such as Nitinol. Also, its manufacture in plastic, silicon, polymers in general or any other malleable material which is not harmful to a patient and which achieves the same technical-functional effects as the guidewire 45 proposed are perfectly acceptable.

In addition to the access trocar 1 previously described, the present invention also includes a method of using an access trocar 1. With reference to FIGS. 1 to 7, the cited method comprises the stage of connecting a first end of a guidewire 45 to an access point 50 on the patient's skin.

In this regard, it is highlighted that the access point 50 must be understood as a site (point) of the skin (also referred to as the epithelial tissue) of the patient where the trocar 1 must be positioned.

To locate and obtain the access point 50, an auxiliary element may be used, such as an 18 Gauge needle, this procedure being preferably, but not exclusively, realized with the assistance of ultrasonographic vision.

Once the access point 50 is established, a first end of the guidewire 45 is connected to the cited access point 50 while a second end of the guidewire 45 must be introduced to the free end 20′ of the trocar 1, as can be better seen in FIG. 5.

Thus, the connection between the first end of the guidewire 45 the access point 50 and the second end of the wire 45 is maintained, and a second end of the wire 45 is introduced into the trocar through its free end 20′.

Subsequently, the method proposed in the present invention also comprises the stage of directly connecting the circlip 21 of the trocar 1 to the epithelial tissue of the patient through the insertion of the guidewire 45 through the hollow portion 25 of the cannula and also through the rod 31 and its orientation duct 33.

So, the guidewire 45 must be introduced at the free end 20′ of the trocar 1 and subsequently inserted through the hollow portion 25 of the cannula 20.

In parallel, the circlip 21 of the trocar 1 must be connected to the epithelial tissue of the patient. In this case, the circlip 21 must be positioned on the patient's skin, but without the cited circlip's going beyond the limits of the skin and being introduced into the patient's body.

Thus, and through the first end of the guidewire 45 which is connected to the access point 50, the medical professional can correctly position the trocar 1 on the patient's skin. In this regard, the guidewire 45 has the function of guiding the medical professional to the correct positioning of the trocar 1 and consequently uses the circlip 21 to anchor the trocar 1 to the patient's skin (but without passing through it). It is emphasized that the stages described above must preferably be realized with the balloon 22 in a deflated (empty) state.

Subsequently, the inflatable balloon 22 must be insufflated with a fluid, ensuring that the correct positioning of the trocar is not affected, in other words, ensuring that the contact of the circlip 21 with the patient's skin is not affected.

In a more specific manner, and in order that the circlip 21 is correctly connected to the patient's skin, it must be inserted in the second end of the guidewire 45 through the cannula 20, the rod 31 and the orientation duct 33 of the trocar 1.

So, it is emphasized that the stage of inserting the guidewire 45 through the rod 31 occurs through the orientation duct 33 positioned inside the rod 31, as can be better observed in FIG. 5. In this way, the guidewire 45 will be fully inserted inside the trocar 1, thus allowing its second end to be visualized by the medical professional on emerging from the dilator structure 30.

Additionally, and with the balloon 22 already insufflated, the first end of the guidewire 45 must be disconnected from the access point 50 and consequently removed from the trocar 1 through the handling portion 40, and, more specifically, through the dilator structure 30. Basically, the guidewire 45 must be pulled and consequently removed from the inside of the trocar 1.

Once these stages have been realized, and using the guidewire 45 as a guide, as described previously, the trocar 1 shall be duly positioned in the body of the patient. Thus, it is possible to remove the handling portion 40 from the trocar 1 and consequently keep the cannula 20 connected to the patient's body.

As such, the trocar 1 will be able to receive, through the hollow portion of the cannula 20, the instruments necessary for the realization of the desired procedures.

In accordance with the previous description, the guidewire 45 used preferably has a diameter in the range of 0.70 mm to 1.0 mm (millimeters) and ideally in the range of 0.85 mm to 1.0 mm. Regarding its length, this is preferably located at 450 mm, but clearly this value may vary depending on the use intended for the trocar 1.

These dimensions allow the guidewire 45 to be duly used to ensure the correct positioning of the trocar 1, thus allowing the guidewire 45 to be inserted through the inside of the cannula 20, the rod 31 and its orientation duct 33.

In this regard, it is highlighted that the diameter of the orientation duct 33 is preferably in the range of 1.0 mm to 2.0 mm and ideally in the range of 1.0 mm to 1.5 mm. As previously observed, it is understood that the diameter of the orientation duct 33 should be slightly greater than the diameter of the guidewire 45, thus allowing the guidewire to be able pass through the inside of the duct 33. In a general sense, it is highlighted that the diameter of the orientation duct 33 is, at a maximum, twice that of diameter of the guidewire 45.

Concerning the diameter of the cannula 20, this must obviously be greater than the diameter of the guidewire 45 and the orientation duct 33. As such, during the insertion of the guidewire 45 through the trocar 1, a kind of “funnel” will be configured between the cannula 20 and the orientation duct 33, thus ensuring that the guidewire 45 is directed to the inside of the duct 33 and that consequently the trocar 1 can be duly positioned in the patient, as will be described below. In general sense, it is proposed that the diameter of the cannula 20 is a maximum of seven times greater than the diameter of the orientation duct 33.

Furthermore, regarding the guidewire 45, this is made from malleable material, preferably metal, such as Nitinol. Also, its manufacture in plastic, silicon, polymers in general or any other malleable material which is not harmful to a patient and which achieves the same technical-functional effects as the guidewire 45 proposed are perfectly acceptable.

Thus, and through the use of the guidewire 45, it is possible to correctly connect the trocar 1 to the patient's body, allowing the trocar 1 to be correctly used for the performance of the desired procedures.

In accordance with trocar 1 and the methodology previously described, the present invention also includes the use of a guidewire 45 for the access trocar, the guidewire 45 comprising a first end opposite a second end.

With reference to FIG. 5, the first end of the guidewire 45 is connected to an access point 50 of an epithelial tissue of a patient, while the second end is introduced to the free end of the trocar 1.

Additionally, the proposed guidewire 45 passes through a cannula 20 and an orientation duct 33 of the trocar, where the guidewire 45 is configured as a malleable rod, preferably metallic (Nitinol) and possesses a diameter preferably in the range of 0.70 mm to 1.0 mm and ideally a diameter in the range of 0.85 mm to 1.0 mm.

The use of the proposed guidewire 45 allows for the correct positioning of the trocar 1 in relation to the epithelial tissue of the patient, since its first end connected to the access point 50 ensures the correct positioning of the trocar 1, allowing, moreover, for its second end to be passed through the inside of the trocar and visualized by the medical professional in the dilator structure 32.

Furthermore, and through the proposed use, the guidewire 45 is able to be fully removed from the trocar 1, through the removal of its first end from the access point 50 thus allowing the first end of the guidewire 45 to be pulled by the medical professional through the inside of the trocar 1.

Finally, the present invention also concerns a kit comprising an access trocar 1 and a guidewire 45, its use being as previously described, thus allowing a medical professional to have to hand all the elements necessary for the correct positioning and use of the trocar 1.

Based on the teachings of the present invention, it comprises an access trocar 1 and its due methodology for the correct positioning of the trocar 1 in the skin of a patient, where a guidewire 45 is used as a guide to position the trocar 1.

Having described an example of a preferred materialization, it must be understood that the scope of the present invention encompasses other possible variations, being limited only by the contents of the attached claims, with the possible equivalents being included therein.

1. An access trocar, the access trocar characterized by the fact that it comprises:
a cannula comprising a hollow portion and further comprising two ends, wherein a first end of the cannula is a free end and the opposite end is a connecting end connectable to a handling portion through a sheath, where:
the handling portion comprises a rod connectable to the hollow portion of the cannula through the connecting end, wherein the handling portion also comprises a dilator structure and a circular structure, wherein, on the introduction of the rod into the hollow portion of the cannula, the circular structure envelops the sheath, and
the cannula also comprises an inflatable balloon and a circlip equipped with internal orifices for insertion of the cannula, wherein the inflatable balloon is also connected to a retention valve through a duct connected to the cannula, wherein the duct is connected to the retention valve through a groove, said groove being positioned at the connecting end of the cannula, wherein the access trocar also comprises:
a guidewire which is inserted into the cannula and the handling portion through the hollow portion and the rod, respectively, wherein the guidewire is configured as a malleable rod which conducts and directs the trocar to its connection to an access point of the epithelial tissue of the patient, wherein the diameter of the guidewire is preferably situated between 0.70 mm to 1.0 mm and more preferably between 0.85 mm to 1.0 mm,
wherein the rod also comprises an orientation duct, the orientation duct comprising a diameter preferably between 1.0 mm to 2.0 mm and more preferably between 1.0 mm to 1.5 mm, wherein the diameter of the orientation duct is, at most, twice that of the diameter of the guidewire and the diameter of the cannula is, at most, seven times that of the diameter of the orientation duct.

2. (canceled)

3. (canceled)

4. A trocar in accordance with claim 1, wherein the inflatable balloon is positioned close to the free end of the cannula and the circlip is positioned between the inflatable balloon and the retention valve.

5. (canceled)

6. (canceled)

7. A trocar in accordance with claim 4, wherein the trocar is able to be connected to an access point through a first end of the guidewire, such that a second end of the guidewire is able to be inserted into the cannula and the rod.

8. A trocar in accordance with claim 7, wherein the handling portion is removable in from the cannula.

9. A trocar in accordance with claim 8, wherein the handling portion comprises an internal diameter compatible with the diameter of the guidewire.

10. A trocar in accordance with claim 9, wherein the rod also comprises an orientation duct, where the orientation duct is configured to allow the guidewire to be passed through its interior.

11. (canceled)

12. (canceled)

13. (canceled)

14. A method of using of a guidewire for an access trocar, the guidewire comprising a first end opposite a second end, wherein the first end is connected to an access point of the epithelial tissue of a patient and the second end is introduced into a free end of the trocar, wherein the guidewire is configured as a malleable rod which conducts and directs the trocar to its connection with the access point of the epithelial tissue of the patient, wherein the guidewire comprises a diameter preferably between 0.70 mm to 1.0 mm and more preferably between 0.85 mm to 1.0 mm, wherein the guidewire is inserted into an cannula and an orientation duct of the trocar, wherein the orientation duct comprises a diameter preferably between 1.0 mm to 2.0 mm and more preferably between 1.0 mm to 1.5 mm, wherein the diameter of the orientation duct is, at most, twice the diameter of the guidewire and the diameter of the cannula is, at most, seven times greater than the diameter of the orientation duct.

15. (canceled)

16. (canceled)

17. A method of using an access trocar, the access trocar comprising a cannula comprising a hollow portion, wherein the cannula also comprises a circlip, the method comprising:
connecting a first end of a guidewire to an access point of the epithelial tissue of a patient;
inserting a second end of the guidewire through the trocar, keeping the first end of the guidewire connected to the access point;
directing the trocar to the access point, using the guidewire as a guide;
disconnecting the first end of the guidewire from the access point; and
removing the guidewire from the trocar, wherein the diameter of the guidewire is preferably situated between 0.70 mm to 1.0 mm and more preferably between 0.85 mm to 1.0 mm,
wherein the inserting the second end of the guidewire through the trocar occurs by means of a rod and an orientation duct positioned inside the rod, wherein the orientation duct comprises a diameter preferably between 1.0 mm to 2.0 mm and more preferably between 1.0 mm to 1.5 mm. wherein the diameter of the orientation duct is, at most, twice that of the guidewire.

18. The method in accordance with claim 17, where the cannula is connected to a handling portion wherein the method also comprises:
removing the handling portion from the trocar.

19. The method in accordance with claim 18, wherein the inserting the second end of the guidewire through the trocar also comprises the inserting the second end of the guidewire through the cannula and the handling portion, keeping the first end of the guidewire connected to the access point.

20. (canceled)

21. The method in accordance with claim 19, wherein the stage of removing the guidewire from the trocar also comprises the stage of inserting the first end of the guidewire through the cannula and the handling portion.

22. (canceled)

23. (canceled)

24. The method in accordance with claim 17, wherein the diameter of the cannula is, at most, seven times greater than the diameter of the orientation duct.

25. A kit comprising an access trocar and a guidewire, wherein the kit comprises an access trocar as defined in claim 1 and a guidewire whose use is as defined in claim 14.

26. The trocar in accordance with claim 10, wherein it is used in pregnant women.

27. The method of using a guidewire for an access trocar in accordance with claim 14, wherein the access trocar is used in pregnant women.

28. The method according to claim 24, wherein the access trocar is used in pregnant women.
